(19) United States
(12) Patent Application Publication
(10) Pub. No.: US 2016/0030206 A1
(43) Pub. Date: Feb. 4, 2016

(54) Liner for Prosthetic Limb

(71) Applicant: UNIVERSITI MALAYA, W.P. Kuala Lumpur (MY)

(72) Inventors: Noor Azuan Abu Osman, W.P. Kuala Lumpur (MY); Hossein Gholizadeh, W.P. Kuala Lumpur (MY)

(73) Assignee: Universiti Malaya, Kuala Lumpur (MY)

(21) Appl. No.: 14/774,711

(22) PCT Filed: Mar. 11, 2014
(86) PCT No.: PCT/MY2014/000030

§ 371 Date: Sep. 11, 2015

(30) Foreign Application Priority Data
Mar. 13, 2013 (MY) PI 2013700414

(51) Int. Cl. A61F 2/78

(52) U.S. Cl. CPC A61F 2/7812; A61F 2230/0069; A61F 2230/0067; A61F 2002/7837; A61F 2002/785; A61F 2002/7818

(57) ABSTRACT

The present invention relates to a liner for limb prosthesis (100), characterized by a foam layer (102) comprising a plurality of strips of foam (102) and a plurality of elastic fabric layers (101), wherein the foam layer (102) is sandwiched between the elastic fabric layers (101) wherein the liner for limb prosthesis (100) comprises a cylindrical or cone shape, with an open end and one close end.

### BACKGROUND OF THE INVENTION

1. Field of the Invention

This invention relates to a liner for upper and lower limb prosthesis using elastic fabric and foam.

2. Description of Related Arts

Prosthesis liner is used for comfort as well as coupling the prosthetic devices to the residual limb for amputees. The interface between the residual limb and the prosthesis is essential for fit and comfort. Perspiration control is a major issue with existing prosthetics and liners as the prior arts are usually airtight, which has several disadvantages.

For example, U.S. Pat. No. 6,136,039 disclosed an invention for a dual durometer silicone liner for prosthesis that has a inner silicone elastomer layer and a relatively harder outer silicone elastomer layer. The liner is formed of a closed end, air tight inner layer of silicone rubber that is highly compliant. However, the elasticity of the liner is limited and it is not reversible.

Furthermore, in U.S. Pat. No. 5,658,354 discloses an invention for lining materials for use with prosthetic devices. The liner is form fitted over the wearer's body comprising of polytetrafluorethylene (PTFE). The interior surface of the PTFE material have been coated with a layer of hydrophilic or oleophobic material such as polyurethane. The coating is to protect the PTFE membrane from compromise due to perspiration. The liner is constructed when two layers of the membrane are formed, each in the outline of that part of the wearer's body over which the non-breathable device is to be worn. The layers are then heat sealed to form a flat, smooth seam along all but one open end of the liner. However, the non-breathable device will cause perspiration to build up, leading to irritation of the skin.

Another example is EP 0870485 B1, is related broadly to the field of prosthetics for a prosthetic liner for an artificial limb to cover and be attached to the stump of an amputee. The specific focus of the invention is a modular concept for providing a custom-fitted liner for prosthesis for an amputee's limb, such as a stump extending downwardly from an amputee's knee or from above the knee. However, the sleeve is not easy to be donned and doffed, as the wearer needs to wear a plurality of layers.

Accordingly, it can be seen in the prior arts that there exists a need to provide a liner for limb prosthesis which is easy to don and doff and have a breathable fabric which reduces the perspiration issue of the prior arts.

### SUMMARY OF INVENTION

It is an objective of the present invention to provide an easily donned and doffed liner for limb prosthesis.

It is also an objective of the present invention to provide a breathable liner for limb prosthesis.

It is yet another objective of the present invention to provide a reversible liner for limb prosthesis.

Accordingly, these objectives may be achieved by following the teachings of the present invention. The present invention relates to a liner for limb prosthesis, characterized by a foam layer comprising a plurality of strips of foam and a plurality of elastic fabric layers, wherein the foam layer is sandwiched between the elastic fabric layers wherein the liner for limb prosthesis comprises a cylindrical or cone shape, with an open end and one close end.

### BRIEF DESCRIPTION OF THE DRAWINGS

The features of the invention will be more readily understood and appreciated from the following detailed description when read in conjunction with the accompanying drawings of the preferred embodiment of the present invention, in which:

FIG. 1 is a diagram showing the first layer of elastic fabric covering the positive cast

FIG. 2 is diagram showing the strips of foam layer.

FIG. 3 is a drawing showing the foam layer sandwiched between the two layers of the elastic fabric and the movement of air between the layers.

### DETAILED DESCRIPTION OF THE INVENTION

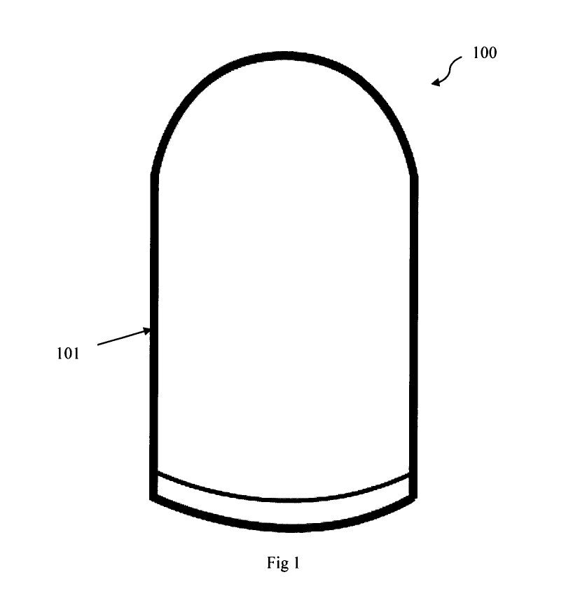

As required, detailed embodiments of the present invention are disclosed herein; however, it is to be understood that the disclosed embodiments are merely exemplary of the invention, which may be embodied in various forms. Therefore, specific structural and functional details disclosed herein are not to be interpreted as limiting but merely as a basis for claims. It should be understood that the drawings and detailed description thereto are not intended to limit the invention to the particular form disclosed, but on the contrary, the invention is to cover all modification, equivalents and alternatives falling within the scope of the present invention as defined by the appended claims. As used throughout this application, the word “may” is used in a permissive sense (i.e., meaning having the potential to), rather than the mandatory sense (i.e., meaning must). Similarly, the words “include,” “including,” and “includes” mean including, but not limited to. Further, the words “a” or “an” mean “at least one” and the word “plurality” means one or more, unless otherwise mentioned. Where the abbreviations or technical terms are used, these indicate the commonly accepted meanings as known in the technical field. For ease of reference, common reference numerals will be used throughout the figures when referring to the same or similar features common to the figures. The present invention will now be described with reference to FIGS. 1-3.

The present invention relates to a liner for limb prosthesis (100), characterized by:
a foam layer (102) comprising a plurality of strips of foam (102);
a plurality of elastic fabric layers (101), wherein the foam layer (102) is sandwiched between the elastic fabric layers (101);
wherein the liner for limb prosthesis (100) comprises a cylindrical or cone shape, with an open end and a close end.

In a preferred embodiment of a liner for limb prosthesis (100), wherein the foam layer (102) comprises foam.

In a preferred embodiment of a liner for limb prosthesis (100) wherein the plurality of elastic fabric layers (101) comprises wool, nylon, cotton, or any combination thereof.

In a preferred embodiment of a liner for limb prosthesis (100), wherein the liner for limb prosthesis (100) is a reversible liner.

### EXAMPLES

Below is an example of a method for preparing the liner for limb prosthesis from which the advantages of the present invention may be more readily understood. It is to be understood that the following example is for illustrative purpose only and should not be construed to limit the present invention in any way.

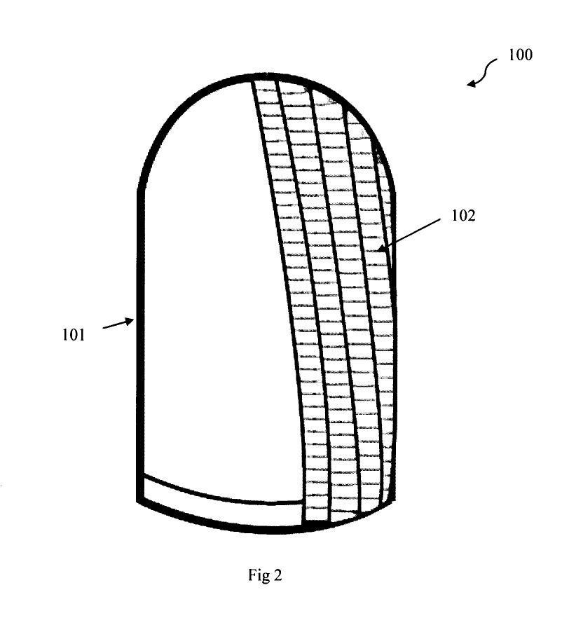

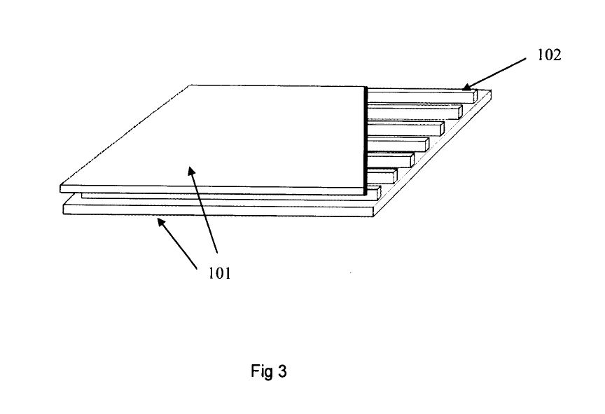

A positive cast of an amputee's residual limb is prepared. A layer of elastic fabric (101), preferably made from nylon, wool or cotton, is placed over the positive cast of the residual limb (FIG. 1). Next, a layer of foam (102) (example, neoprene) is laid over the first layer of elastic fabric. The layer of foam (102) is cut into uniform strips (FIG. 2). This allows the liner (100) to be both elastic and flexible. Another layer of elastic fabric (101) is then placed over the layer of foam (102) (FIG. 3). The three layers comprising the foam strips (102) and elastic fabrics (101) are then sewn together using a sewing machine to form a unitary prosthetic limb liner (100).

The method used will fabricate a liner for limb prosthesis (100) that has enhanced elasticity, flexibility and shock absorption ability. It is also more comfortable to wear as it has less friction and also provides a cool environment for the residual limb. The shape of the liner (100) is adapter to the dimensions of the user's residual limb, preferably in cylindrical or cone shape. The residual limb is inserted into the liner (100) through the open end of said liner (100). Due to the elastic and flexible nature of the liner (100), no straps or fasteners are required to secure the liner (100). The limb prosthesis is then worn over the liner (100), with the close end of the liner (100) cushioning the residual limb against the liner (100). One advantage of the developed liner (100) is that it is a reversible liner, where both the internal and external surfaces of the liner (100) can be used. The material used in the fabrication of the liner (100) allows the fabric to be breathable, allowing air to easily move inside the liner (100).

Although the present invention has been described with reference to specific embodiments, also shown in the appended figures, it will be apparent for those skilled in the art that many variations and modifications can be done within the scope of the invention as described in the specification and defined in the following claims.

Description of the reference numerals used in the accompanying drawings according to the present invention:

| Reference | |
|---|---|
| Numerals | Description |
| 100 | Liner for limb prosthesis |
| 101 | Elastic fabric layer |
| 102 | Foam layer |

I/we claim:

1. A liner for limb prosthesis (100), characterized by:
a foam layer (102) comprising a plurality of strips of foam (102);
a plurality of elastic fabric layers (101), wherein the foam layer (102) is sandwiched between the elastic fabric layers (101);
wherein the liner for limb prosthesis(100) comprises a cylindrical or cone shape, with an open end and a close end.

2. A liner for limb prosthesis (100) according to claim 1, wherein the foam layer (102) comprises foam.

3. A liner for limb prosthesis (100) according to claim 1, wherein the plurality of elastic fabric layers (101) comprises wool, nylon, cotton, or any combination thereof.

4. A liner for limb prosthesis (100) according to claim 1, wherein the liner for limb prosthesis (100) is a reversible liner.
